(19) United States
(12) Patent Application Publication
(10) Pub. No.: US 2025/0083137 A1
(43) Pub. Date: Mar. 13, 2025

(54) SUPPORTED HYDROFORMYLATION CATALYST AND PREPARATION METHOD THEREOF, AND METHOD FOR SYNTHESIZING 4-ACETYLOXY-2-METHYL-2-BUTENAL

(71) Applicant: Shaoxing University, Shaoxing (CN)

(72) Inventors: Xuemei Wei, Shaoxing (CN); Guoqi Yu, Hangzhou (CN); Hualiang Shen, Shaoxing (CN); Tao Cai, Shaoxing (CN); Yanjuan Luo, Shaoxing (CN); Tianbo Shang, Shaoxing (CN); Runpu Shen, Shaoxing (CN); Huiting Xu, Shaoxing (CN); Chunlei Wu, Shaoxing (CN); Kai Wang, Shaoxing (CN)

(21) Appl. No.: 18/503,250

(22) Filed: Nov. 7, 2023

(30) Foreign Application Priority Data
Sep. 12, 2023 (CN) 2023111749022

(51) Int. Cl. B01J 31/28; B01J 21/18; B01J 23/46; B01J 31/24; B01J 37/02; B01J 37/08; B01J 37/18; B01J 37/34; C07C 67/38

(52) U.S. Cl. CPC B01J 31/28; B01J 21/18; B01J 23/464; B01J 31/2442; B01J 37/0201; B01J 37/0236; B01J 37/086; B01J 37/18; B01J 37/343; C07C 67/38; B01J 2231/321; B01J 2531/004; B01J 2531/822

(57) ABSTRACT

A supported hydroformylation catalyst and an application method thereof, and a method for synthesizing a 4-acetyloxy-2-methyl-2-butenal are provided. A structure expression of the supported hydroformylation catalyst is Rh@P—N/AC, Rh of which represents an active center of the catalyst and P—N/AC of which represents carriers of the catalyst including an AC carrier and a P—N carrier. The AC carrier in the P—N/AC is an activated carbon and the P—N carrier in the P—N/AC is a phosphine-nitrogen (P—N) organic ligand with one structural formula selected from the group consisting of structural formulas I-IV. The catalyst is capable of achieving catalytic cycling of precious metal and phosphine ligand and has high activity and C₂ selectivity.

### TECHNICAL FIELD

The disclosure relates to the field of homogeneous catalyst heterogenization technologies, particularly to a phosphine-nitrogen (P—N) organic ligand, a supported hydroformylation catalyst and a preparation method thereof, and a method for synthesizing a 4-acetyloxy-2-methyl-2-butenal.

### BACKGROUND

The 4-acetyloxy-2-methyl-2-butenal is a chemically synthesized product and a structural formula thereof is as follows:

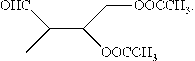

The 4-acetyloxy-2-methyl-2-butenal is an intermediate of vitamin A and plays an important role in an industry of synthesizing the vitamin A. Therefore, a research on a synthesis process of the 4-acetyloxy-2-methyl-2-butenal has always been a research hotpot.

At present, a preparation process of an aldehyde group mainly adopts cobalt-based (cobalt abbreviated as Co) homogeneous/heterogeneous catalysts and rhodium-based (rhodium abbreviated as Rh) homogeneous/heterogeneous catalysts. Reaction conditions of a cobalt-based catalytic system are relatively harsh, requiring high temperature and high pressure. However, a homogeneous Rh—P (phosphorus) catalytic system has advantages of mild reaction conditions and high reaction activity, thereby being widely researched in industry.

The US patent NO. U.S. Pat. No. 10,913,055B2 uses a chemical compound represented by the following formula as a phosphine ligand to catalyze hydroformylation at a temperature of 70-90 degrees Celsius (° C.) under a pressure of 14-20 bar:

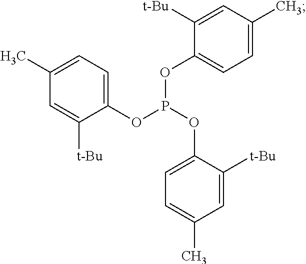 [SMILES: Cc1ccc(OP(Oc2ccc(C)cc2C(C)(C)C)Oc2ccc(C)cc2C(C)(C)C)c(C(C)(C)C)c1]

a reaction conversion rate of the hydroformylation thereof is greater than 61.9%; and a ratio between linear (normal) and branched (iso) aldehydes (also referred as to an n/i ratio) thereof is in a range of 1.09 to 1.91. The US patent NO. U.S. Pat. No. 6,362,354B1 uses a chemical compound represented by the following formula as a phosphonite ligand:

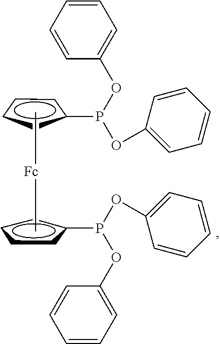

which uses Rh (acetylacetonate) (CO) 2 as a central metal to catalyze hydroformylation of butadience at a temperature of 95° C. under a pressure of 0.69 mega Pascal (MPa), thereby realizing an efficient reaction conversion. China patent application NO. CN115739184A uses a specific combination of a phosphoramidite monophosphine ligand and a phosphoramidite diphosphine ligand as a ligand and uses a P—N structure with strong x electron receiving capability to enhance an electron receiving capability and a space effect of the Rh and to catalyze hydroformylation of dipolyisobutylene, thereby increasing catalytic activity of the hydroformylation with a reaction conversion rate greater than 95%. However, a homogeneous catalytic system has inherent drawbacks, such as difficulty in catalyst regeneration and circulation, poor selectivity, complex separation and purification processes, and requirement of adding an organic phosphine ligand in the homogeneous catalytic system, which puts forwards higher requirements on production equipment, significantly increases production costs while posing new challenges to the ecological environment, and seriously restricts large-scale industrial production.

China patent application NO. CN115739146A provides a preparation method of a heterogeneous hydroformylation catalyst, including: preparing an active metal Rh into a ferromagnetic intermediate Fe304@SiO₂—NH₂—Rh, then adding polyvinylpyrrolidone as an active agent to prepare a core-shell catalyst Fe304@SiO₂—NH₂—Rh@SiO₂, and etching core shells by using a sodium hydroxide solution to obtain the heterogeneous hydroformylation catalyst; and the heterogeneous hydroformylation catalyst is applied to the hydroformylation of olefins in FCC light gasoline and F-T synthetic distillate oil. China patent application NO. CN115041232A discloses a preparation method of a heterogeneous hydroformylation catalyst, which is constituted of a phosphine-containing organic porous copolymer supported with metal Rh; and the phosphine-containing organic porous copolymer is formed by copolymerization of at least one monodentate phosphite monomer and at least one bidentate phosphite monomer. Furthermore, the prepared heterogeneous hydroformylation catalyst exhibits excellent catalytic performance in the hydroformylation of mixed olefins and exhibits good reactivity towards C₁ selectivity (referred as to C₁ product selectivity of the catalyst). China patent application NO. CN113856721A discloses a heterogeneous Rh—CoO—NNTs catalyst with a synergistic effect between Rh—CoO and Rh—NNTs, catalytic activity and stability of which exceed those of Rh—CoO catalyst, thereby exhibiting good catalytic activity and stability in the hydroformylation conversion of cycloolefins. China patent application NO. CN114931961A provides a preparation method for an immobilized rhodium-based catalyst, and the method includes using a transition metal phosphide supported rhodium as the catalyst and adding a diphosphine ligand; and then the obtained catalytic system has high catalytic activity and stability, with a C₁ selectivity over 87.6% and a conversion rate of over 99%. However, the above obtained catalytic systems have not yet achieved the circulation of the phosphine ligand.

### SUMMARY

In view of the above, objectives of the disclosure are to provide a supported hydroformylation catalyst and a preparation method thereof, and a method for synthesizing a 4-acetyloxy-2-methyl-2-butenal. The supported hydroformylation catalyst provided by the disclosure is capable of achieving catalytic cycling of precious metal and phosphine ligand, and has a high activity and C₂ selectivity (referred as to a C₂ product selectivity of the catalyst).

In order to achieve the above objectives, the disclosure provides a technical solution as follows: providing a supported hydroformylation catalyst, a structure expression of which is Rh@P—N/AC; in the structure expression of the supported hydroformylation catalyst, Rh represents rhodium (Rh) atoms that are used to be an active center of the supported hydroformylation catalyst and P—N/AC represents carriers of the supported hydroformylation catalyst including an AC carrier and a P—N carrier; and the AC carrier in the P—N/AC is an activated carbon; and the P—N carrier in the P—N/AC is a phosphine-nitrogen (P—N) organic ligand with one structural formula selected from the group consisting of the following structural formulas I-IV:

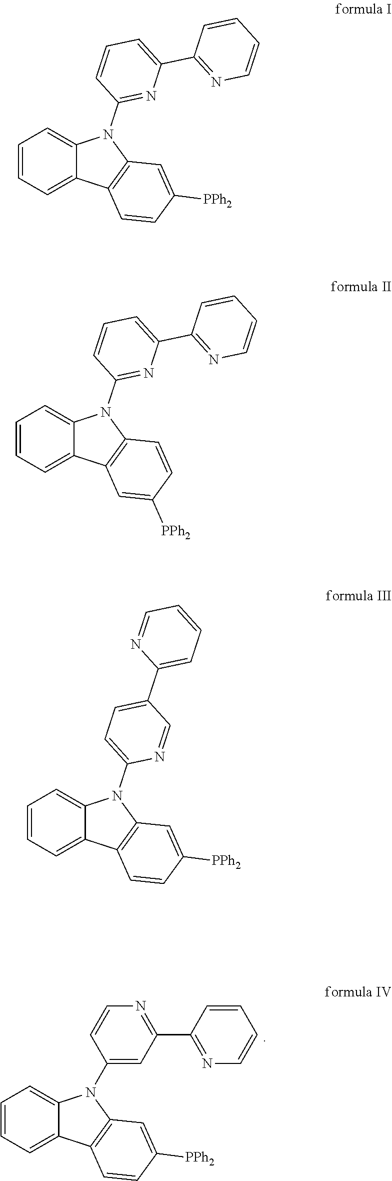

In an embodiment, a particle size of each of the Rh atoms is in a range of 1-3 nanometers (nm); a supported capacity of the Rh atoms is in a range of 2% to 5%; and the activated carbon is powdered with a specific surface in a range of 800 square meters per gram (m²/g) to 1,000 m²/g.

In an embodiment, a preparation method of the P—N organic ligand includes the following steps:
performing a coupled reaction on a 2-bromocarbazole and a bipyridine bromide to obtain an N-bipyridine carbazole intermediate, and then performing a couple reaction on the N-bipyridine carbazole intermediate and a chlorodiphenyl phosphine to obtain the P—N organic ligand.

The disclosure further provides a preparation method of the supported hydroformylation catalyst described in the above technical solution, including the following steps:
step 1, performing pre-coordination on the P—N organic ligand and a Rh precursor to obtain a mixed solution;
step 2, adding the activated carbon to the mixed solution obtained in the step 1, performing ultra-sonication on the mixed solution added with the activated carbon to disperse the carriers and obtaining a solid catalyst sample by impregnation, and rotary evaporating and drying the solid catalyst sample to obtain a solid sample; and
step 3, performing hydrogenation reduction and roasting carbonization on the solid sample obtained in the step 2 at a temperature of 500 degrees Celsius (° C.) to 700° C. with a heating rate of 5-10° C. per minute (° C./min), thereby obtaining the supported hydroformylation catalyst.

In an embodiment, in the step 1, a molar ratio of Rh element in the Rh precursor to the P—N organic ligand is (0.5-10): 1.

In an embodiment, the Rh precursor is a rhodium chloride trihydrate or a dicarbonylacetylacetonato rhodium.

In an embodiment, reaction conditions of the pre-coordination in the step 1 include: a room temperature, an atmospheric pressure, and a reaction time in a range of 2-5 hours (h).

In an embodiment, a time for the roasting carbonization is 2 h.

The disclosure further provides a method for synthesizing the 4-acetyloxy-2-methyl-2-butenal, including: performing hydroformylation of a but-3-ene-1,2-diyl diacetate by using the Rh@P—N/AC supported hydroformylation catalyst to obtain the 4-acetyloxy-2-methyl-2-butenal.

Beneficial effects of the disclosure are as follows.
1. The heterogeneous catalyst (also referred as to the supported hydroformylation catalyst) provided by the disclosure is used to catalyze the preparation of the 4-acetyloxy-2-methyl-2-butenal with a conversion rate over 98% and a C₂ selectivity of the disclosure reaches 80%. Compared with recorded industrial production data, the disclosure has significantly improved.
2. The disclosure adopts the heterogeneous catalyst without additionally adding the organic phosphine ligand, the catalytic system cycle can be successfully achieved, which has good stability and meets requirements of green production for enterprises.

### BRIEF DESCRIPTION OF DRAWINGS

FIG. 1 illustrates an X-ray diffraction (XRD) spectrum of an Rh@P—N/AC catalyst with a supported capacity of Rh atoms being 5% according to an embodiment 1 of the disclosure.

FIG. 2A illustrates a high resolution transmission electron microscope (HRTEM) image of the Rh@P—N/AC catalyst with the supported capacity of Rh atoms being 5% according to the embodiment 1 of the disclosure.

FIG. 2B illustrates a schematic diagram of a mapping of the Rh@P—N/AC catalyst with the supported capacity of Rh atoms being 5% according to the embodiment 1 of the disclosure.

FIG. 3 illustrates a schematic diagram of a reaction mechanism of a catalytic hydroformylation reaction of a but-3-ene-1,2-diyl diacetate for preparing a 4-acetyloxy-2-methyl-2-butenal according to an embodiment 2 of the disclosure.

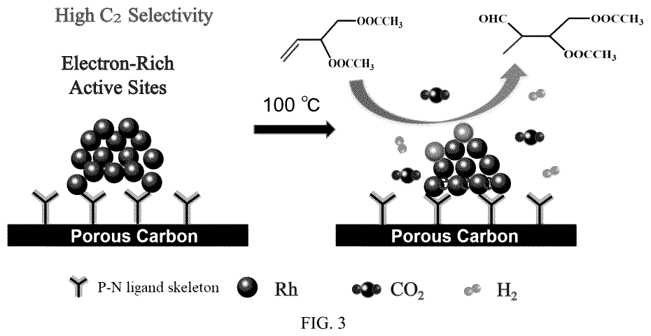

### DETAILED DESCRIPTION OF EMBODIMENTS

A preparation of a traditional hydroformylation heterogeneous catalyst uses an activated carbon as a carrier and an additional phosphine ligand is added therein. The prepared catalyst has a weak interaction on a chemical bond between rhodium (Rh) atoms and carbon (C) atoms, resulting in easy loss of the Rh atoms, and cannot yet be applied in industry. Introducing elements of nitrogen (N) and phosphorus (P) can effectively improve properties of the activated carbon, but a traditional physical doping method has problems such as uneven distribution of the elements, weak interaction strength among the elements, easy loss of the elements, and there is a need to add an additional organic ligand in view of the traditional physical doping method. However, a catalyst obtained by a preparation method of the disclosure is rich in phosphine-nitrogen (P—N) functional groups on its surface, and the P—N functional groups are evenly dispersed. At the same time, a high dispersion and efficient utilization of Rh atoms in the catalyst of the disclosure are achieved by utilizing a multi-dentate coordination between the Rh atoms and the P—N organic ligand, thereby ensuring a basic activity and stability of the catalyst of the disclosure. When a specific organic ligand is determined, there is no need to additionally add a phosphine ligand, and a catalytic cycle of the organic ligand is successfully achieved with a high C₂ selectivity (referred as to a C₂ product selectivity of the catalyst).

Based on the above principles, the disclosure provides a supported hydroformylation catalyst. In the disclosure, rhodium atoms are regarded as an active center, and a key point of the disclosure lies in a selection and immobilization of the P—N organic ligand and suitable adjustments on electrons and structure, thereby realizing high activity of the Rh atoms and high C₂ selectivity of the catalyst.

In the disclosure, a structure expression of the supported hydroformylation catalyst is Rh@P—N/AC, Rh of which represents Rh atoms that are used to be an active center of the supported hydroformylation catalyst and P—N/AC of which represents carriers of the supported hydroformylation catalyst including an AC carrier and a P—N carrier; and the AC carrier in the P—N/AC is an activated carbon and the P—N carrier in the P—N/AC is a P—N organic ligand with one structural formula selected from the group consisting of the following structural formulas I-IV:

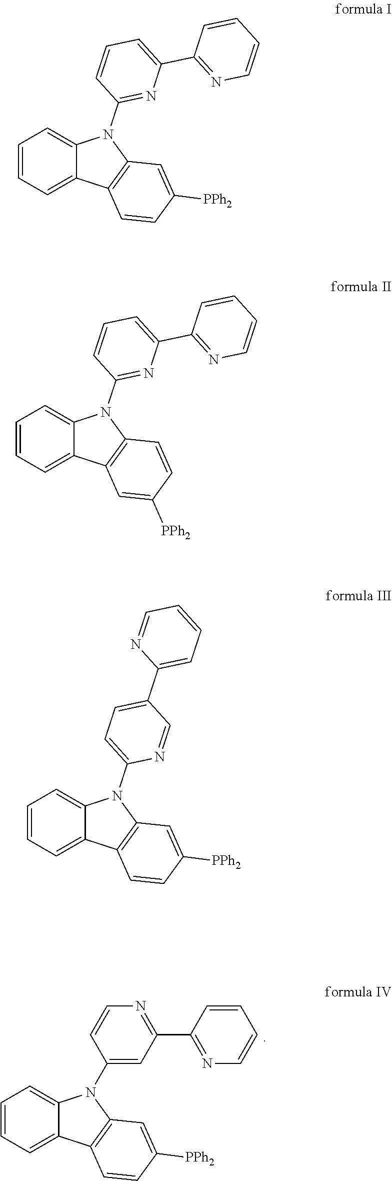

In some embodiments, a particle size of each of the Rh atoms is in a range of 1-3 nanometers (nm); a supported capacity of the Rh atoms is in a range of 2% to 5%; and the activated carbon is powdered with a specific surface in a range of 800 square meters per gram (m²/g) to 1,000 m²/g. The Rh particle size in the range of 1-3 nm can significantly improve the basic activity and stability of the center Rh metal. The activated carbon plays an important role in the hydroformylation reaction, promotes or strengthens an adsorption of carbon monoxide (CO), and inhibits dissociation of CO. Furthermore, a rich pore structure in the activated carbon is conducive to diffusion and mass transfer of reaction materials and products, making it more suitable for the hydroformylation reaction of olefins.

In some embodiments, a preparation method of the P—N organic ligand includes the following steps:
performing a coupled reaction on a 2-bromocarbazole and a bipyridine bromide to obtain an N-bipyridine carbazole intermediate, and then performing a couple reaction on the N-bipyridine carbazole intermediate and a chlorodiphenyl phosphine to obtain the P—N organic ligand.

Specially, a chemical compound represented by the following structural formula is synthesized as an example of the P—N organic ligand:

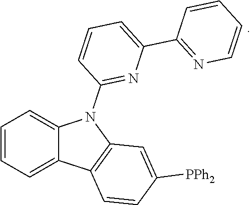

And then, a synthesis process of the above chemical compound is as follows:

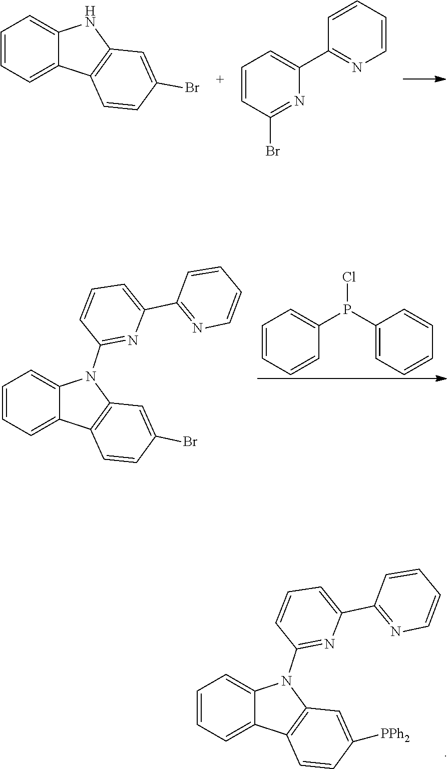

Furthermore, a preparation method of the above P—N organic ligand (also referred as to the chemical compound) includes the following steps:
step 1 of a coupled reaction on the 2-bromocarbazole and the bipyridine bromide, including: adding 5.39 grams of the 2-bromocarbazole in 22 millimoles (mmol), 50 milliliters (mL) of 1,3-dimethyl-2-imidazolidinone, 5 g of the bipyridine bromide in 20 mmol, 0.19 g of cuprous iodide in 1 mmol, and 1.76 g of lithium tert-butoxide in 22 mmol (equal to 1 equivalent abbreviated as equiv) in a 150 mL sealed tube to obtain a system, and then stirring the system at 150 degrees Celsius (° C.) to perform a reaction on the system for 16 hours (h), tracking a reaction process of the system by using a thin-layer chromatography (TLC) plate with a ratio of ethyl acrylate (EA) to polyethylene (PE) of being 1:10 and a Rf (referred as to a proportion between a distance moved from origin by component and a distance moved from origin by solvent) of being 0.3; after the reaction, using an oil pump to perform a decompression distillation at 100° C. to remove a brown oily substance of the 1,3-dimethyl-2-imidazolidinone; thereafter adding 25 ml of ethyl acetate and 200 ml of purified water to the system, and stirring the system at room temperature for 15 minutes (min) to precipitate a large amount of light yellow solid, filtrating the large amount of light yellow solid to obtain a large amount of solid crude product, and then obtaining the N-bipyridine carbazole intermediate separated by column chromatograph; and
step 2 of a synthesis of the P—N organic ligand, including: under a temperature of −78° C. by using a cold trap and nitrogen protection, slowly add 10 mL of tetrahydrofuran into 0.6 g of the N-bipyridine carbazole intermediate in 1.5 mmol prepared in the step 1, and then slowly dripping 0.66 mL of n-butyl lithium in 1.1 equiv dissolved in 2.5 moles per litter (mol/L) of cyclohexane, thereafter stirring the system at the temperature of −78° C. for 3 h; and then, dripping 2 ml of tetrahydrofuran solution with 0.366 g of diphenylphosphonium chloride in 1.65 mmol; after the dripping, turn off the cold trap to slowly rise the temperature to room temperature and stirring the system overnight to perform a synthesis reaction; after the synthesis reaction is completed, adding an appropriate amount of methanol to terminate the synthesis reaction to obtain a product. Moreover, the product is performed by decompression distillation to remove organic solvents thereon, and then is extracted by ethyl acetate and purified water to obtain an organic layer of the product, thereafter concentrating the organic layer to obtain a crude product, and finally, the P—N organic ligand is obtained from purifying the crude product by column chromatography with EA: PE of being 1:10.

Furthermore, the disclosure further provides a preparation method of the supported hydroformylation catalyst obtained from the above technical solution, including the following steps:
step 1, performing pre-coordination on the P—N organic ligand and a Rh precursor to obtain a mixed solution;
step 2, adding the activated carbon to the mixed solution obtained in the step 1, performing ultra-sonication on the mixed solution added with the activated carbon to disperse the carriers and obtaining a solid catalyst sample by impregnation, and rotary evaporating and drying the solid catalyst sample to obtain a solid sample; and
step 3, performing hydrogenation reduction and roasting carbonization on the solid sample obtained in the step 2 at a temperature of 500° C. to 700° C. with a heating rate of 5-10° C. per minute (° C./min), thereby obtaining the supported hydroformylation catalyst.

In the disclosure, a molar ratio of Rh element in the Rh precursor to the P—N organic ligand is (0.5-10): 1. In an illustrated embodiment, the molar ratio is 4:1, which can guarantee the dispersion of the Rh atoms to achieve maximum atomic utilization, without resulting in a decrease in the basic activity of the catalyst because of the strong binding between the metal and the carriers.

In the disclosure, the Rh precursor is a rhodium chloride trihydrate or a dicarbonylacetylacetonato rhodium. In an illustrated embodiment, the Rh precursor is the dicarbonylacetylacetonato rhodium.

In the disclosure, the P—N organic ligand and the Rh precursor are pre-mixed in ethanol solution and then are stirred evenly at room temperature for 2-5 h, thereby performing the pre-coordination.

In the preparation method of the catalyst of the disclosure, before adding the activated carbon to the mixed solution obtained in the step 1, the activated carbon is pre-processed by pulverization; after the pulverization, a mesh number of the activated carbon is in a range of 100-300 mesh. In an illustrated embodiment, the activated carbon is graphitized activated carbon. Moreover, a time for the ultra-sonication in the step 2 is preferably 2 h; a temperature for the drying in the step 2 is preferably 60° C.; and a time for the drying in the step 2 is preferably 8-12 h.

In the disclosure, the hydrogenation reduction and the roasting carbonization are key points in the preparation of the catalyst of the disclosure. While performing the reduction on the Rh atoms, the P—N organic ligand needs to be carbonized to obtain specific P—N groups, which greatly depends on the reduction temperature and the heating rate. When the reduction temperature is too low, the carbonization will be incomplete and the organic ligand and the central metal will lose. And when the reduction temperature is too high, the central mental will agglomerate and metal utilization rate is decrease. Therefore, in an illustrated embodiment, the temperature of the roasting carbonization is 550° C.

The disclosure further provides a method for synthesizing a 4-acetyloxy-2-methyl-2-butenal, including: performing hydroformylation of a but-3-ene-1,2-diyl diacetate by using the Rh@P—N/AC supported hydroformylation catalyst to obtain the 4-acetyloxy-2-methyl-2-butenal. Specially, the supported hydroformylation catalyst prepared by the disclosure and raw materials of the but-3-ene-1,2-diyl diacetate and toluene are added to a high-pressure reactor, and then synthesis gas is introduced into the high-pressure reactor to perform the hydroformylation reaction on the supported hydroformylation catalyst and the raw materials. In the disclosure, the synthesis gas is a mixture of CO and hydrogen (H2); a temperature of the hydroformylation reaction is in a range of 60° C. to 120° C. and in an illustrated embodiment, the temperature is 100° C.; a time of the hydroformylation reaction is in a range of 4 h to 20 h and in an illustrated embodiment, the time is 10 h; and a pressure of the hydroformylation reaction is in a range of 5 MPa to 12 MPa and in an illustrated embodiment, the pressure is 8 MPa.

In order to better understand the disclosure, the content of the disclosure is further elucidated in conjunction with embodiments, but the content of the disclosure is not limited to the following embodiments. All of technical solutions implemented based on the above content of the disclosure are covered within the scope of the protection intended by the disclosure. Unless otherwise specified, the raw materials and reagents used in the following embodiments are commercially available or can be prepared by known methods.

Purchase of the raw materials is as follows.

The activated carbon is purchased from Xi'an Kaili New Materials Co., Ltd., in powder form with a specific surface area of 800-1,000 m²/g.

The rhodium chloride trihydrate and the dicarbonylacetylacetonato rhodium are purchased from Shaanxi Kaida Chemical Co., Ltd. with an each purity of 99%.

All of a 1-bromo-9H-carbazole, a 6-bromo-2,2′-bipyridine, a chlorodiphenyl phosphine, and a but-3-ene-1,2-diyl diacetate are purchased from Aladdin each with analytical reagent (AR) purity.

A molar ratio of CO: H2 in the synthetic gas is 1:1, a purity of the synthetic gas is 99.9%, a sulfur(S) content of the synthetic gas is smaller than 1 part per million (ppm), and the synthetic gas is prepared by an experimenter.

And the reagents are with analytical reagent purities.

### Embodiment 1

A P—N organic ligand represented by the following structural formula is provided:

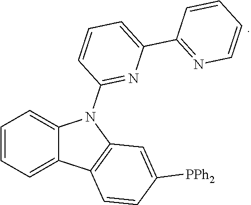

and a preparation method of the P—N organic ligand includes the following steps:
step 1 of a coupled reaction on a 2-bromocarbazole and a bipyridine bromide, including: adding 5.39 grams of the 2-bromocarbazole in 22 millmoles (mmol), 50 milliliters (mL) of 1,3-dimethyl-2-imidazolidinone, 5 g of a 6-bromo-2,2′-bipyridine in 20 mmol, 0.19 g of cuprous iodide in 1 mmol, and 1.76 g of lithium tert-butoxide in 22 mmol (1 equiv) in a 150 mL sealed tube to obtain a system, and then stirring the system at 150° C. to perform a reaction on the system for 16 h, tracking a reaction process of the system by using a TLC plate with a ratio of EA: PE of being 1:10 and a Rf of being 0.3; after the reaction, using an oil pump to perform a decompression distillation at 100° C. to remove a brown oily substance of the 1,3-dimethyl-2-imidazolidinone; thereafter adding 25 ml of ethyl acetate and 200 ml of purified water to the system, and stirring the system at room temperature for 15 min to precipitate a large amount of light yellow solid, filtrating the large amount of light yellow solid to obtain a large amount of solid crude product, and then obtaining the N-bipyridine carbazole intermediate separated by column chromatograph; and
step 2 of a synthesis of the P—N organic ligand, including: under a temperature of −78° C. by using a cold trap and nitrogen protection, slowly add 10 mL of tetrahydrofuran into 0.6 g of the N-bipyridine carbazole intermediate in 1.5 mmol prepared in the step 1, and then slowly dripping 0.66 mL of n-butyl lithium in 1.1 equiv dissolved in 2.5 mol/L of cyclohexane, thereafter stirring the system at the temperature of −78° C. for 3 h; and then, dripping 2 ml of tetrahydrofuran solution with 0.366 g of diphenylphosphonium chloride in 1.65 mmol; after the dripping, turn off the cold trap to slowly rise the temperature to room temperature and stirring the system overnight to perform a synthesis reaction; after the synthesis reaction is completed, adding an appropriate amount of methanol to terminate the synthesis reaction to obtain a product. Moreover, the product is performed by decompression distillation to remove organic solvents thereon, and then is extracted by ethyl acetate and purified water to obtain an organic layer of the product, thereafter concentrating the organic layer to obtain a crude product, and finally, the P—N organic ligand is obtained from purifying the crude product by column chromatography with EA: PE of being 1:10.

An Rh@P—N/AC catalyst A with a supported capacity of Rh atoms being 5% is prepared, including the following steps:
step 1, pre-mixing the dicarbonylacetylacetonato rhodium with the P—N organic ligand prepared in the embodiment 1 in a molar ratio of rhodium element in the dicarbonylacetylacetonato rhodium to the P—N organic ligand being 2:1 in an ethanol solution, and then stirring the ethanol solution mixed with the dicarbonylacetylacetonato rhodium and the P—N organic ligand at room temperature for 4 h to perform pre-coordination, thereby obtaining a mixed solution;
step 2, adding 1 g of activated carbon to the mixed solution obtained in the above step 1, performing ultra-sonication on the mixed solution added with the activated carbon for 2 h to disperse the carriers (i.e., the AC carrier and the P—N carrier) and obtaining a solid catalyst sample by impregnation, and rotary evaporating and drying the solid catalyst sample at 60° C. for 10 h to obtain a solid sample; and
step 3, performing a reduction on the solid sample in a hydrogen atmosphere and then calcining (also referred as to roasting carbonization) the solid sample at 550° C. with a heating rate of 5° C./min, and then obtaining a target product (also referred as to the Rh@P—N/AC catalyst A).

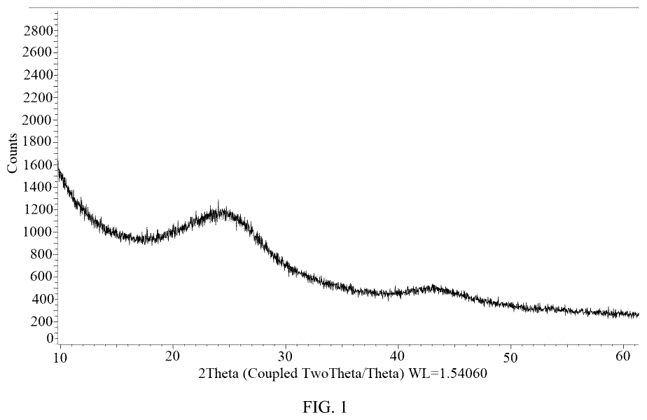

The Rh@P—N/AC catalyst A with the supported capacity of Rh atoms being 5% is characterized by an X-ray diffraction (XRD) spectrum as shown in FIG. 1. With reference to FIG. 1, it can be seen that a carrier of the Rh@P—N/AC catalyst A is an amorphous carbon carrier and a structure of the carbon carrier is not changed after being treated with the P—N organic ligand. Furthermore, there is an absence of Rh characteristic peak in the XRD, indicating a high fraction of Rh in the Rh@P—N/AC catalyst A.

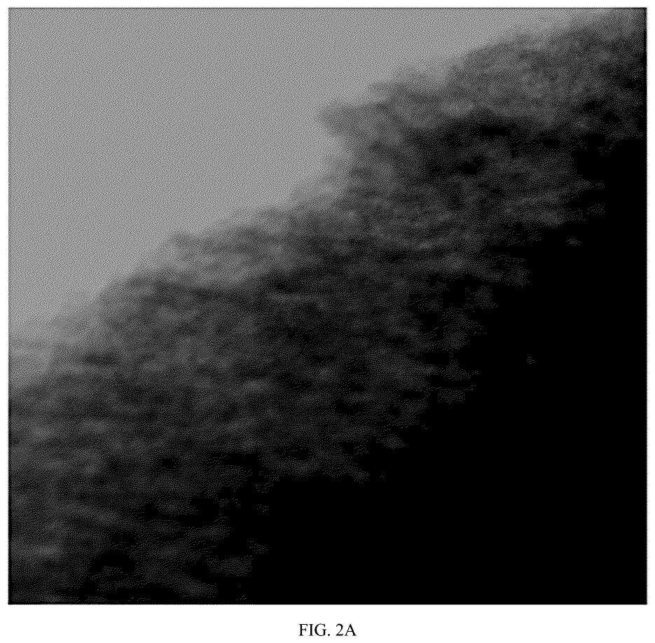

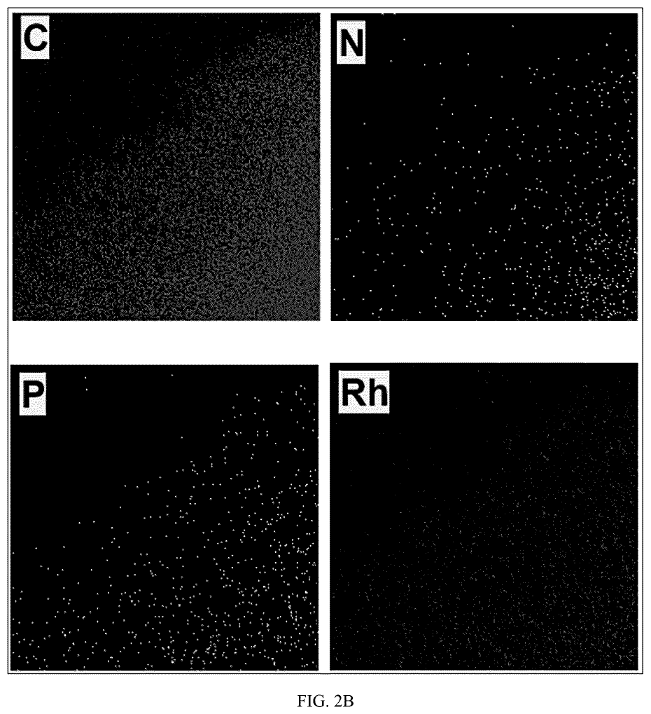

According to a mapping of the Rh@P—N/AC catalyst A with the supported capacity of Rh atoms being 5% as shown in FIG. 2B, four elements of carbon (C), nitrogen (N), phosphorus (P), and rhodium (Rh) are detected on the surface of the Rh@P—N/AC catalyst A; the P—N organic ligand is uniformly dispersed on the surface of the activated carbon carrier; the rhodium element is uniformly dispersed without agglomeration phenomenon.

### Embodiment 2

A 4-acetyloxy-2-methyl-2-butenal is prepared from catalyzing hydroformylation of a but-3-ene-1,2-diyl diacetate by using the Rh@P—N/AC catalyst A and a preparation process of the 4-acetyloxy-2-methyl-2-butenal includes the following steps:
taking 1 mmol of the Rh@P—N/AC catalyst A prepared in the embodiment 1 and raw materials of 0.1 mol of the but-3-ene-1,2-diyl diacetate and 10 mL of toluene to add into a 100 mL high pressure reactor, introducing the synthetic gas to react the Rh@P—N/AC catalyst A and the raw materials at 10 MPa and 100° C. for 10 h to obtain a reaction product; and sampling the reaction product to analyze, finding that a conversion rate of the raw materials reaches 99.0% and C₂ selectivity reaches 80.4%.

### Embodiment 3

An Rh@P—N/AC catalyst B with a supported capacity of Rh atoms being 5% is prepared.

Compared with the embodiment 1, a difference is that the dicarbonylacetylacetonato rhodium in the step 1 is changed to an equal amount of a rhodium chloride trihydrate.

### Embodiment 4

An Rh@P—N/AC catalyst C with a supported capacity of Rh atoms being 5% is prepared.

Compared with the embodiment 1, a difference is that a molar ratio of the dicarbonylacetylacetonato rhodium to the P—N organic ligand in the step 1 is replaced by 5:1.

### Embodiment 5

An Rh@P—N/AC catalyst D with a supported capacity of Rh atoms being 5% is prepared.

Compared with the embodiment 1, a difference is that a molar ratio of the dicarbonylacetylacetonato rhodium to the P—N organic ligand in the step 1 is replaced by 1:1.

### Embodiment 6

An Rh@P—N/AC catalyst E with a supported capacity of Rh atoms being 5% is prepared.

Compared with the embodiment 1, a difference is that a molar ratio of the dicarbonylacetylacetonato rhodium to the P—N organic ligand in the step 1 is replaced by 0.5:1.

### Embodiment 7

An Rh@P—N/AC catalyst F with a supported capacity of Rh atoms being 5% is prepared.

Compared with the embodiment 1, a difference is that a temperature of the calcining in the step 3 is 700° C.

### Embodiment 8

An Rh@P—N/AC catalyst G with a supported capacity of Rh atoms being 5% is prepared.

Compared with the embodiment 1, a difference is that the heating rate in the step 3 is 10° C./min.

### Control Example 1

An Rh/AC catalyst with a supported capacity of Rh atoms being 5% is prepared.

Compared with the embodiment 1, a difference is that in the step 1, there is no addition of a P—N organic ligand.

### Control Example 2

A 4-acetyloxy-2-methyl-2-butenal is prepared from catalyzing hydroformylation of a but-3-ene-1,2-diyl diacetate by using the Rh/AC catalyst and a preparation process of the 4-acetyloxy-2-methyl-2-butenal includes as the embodiment 2; and a difference between the embodiment 2 and the control example 2 is that the Rh@P—N/AC catalyst A is replaced by the Rh/AC catalyst with the supported capacity of Rh atoms being 5%.

The reaction product is sampled to analyze, finding that the reaction of the control example 2 is an equivalent reaction. Therefore, it indicates that catalytic cycling cannot be achieved in the control example 2 without adding the phosphine ligand and only can the equivalent reaction occur. However, the embodiment 1 using the Rh@P—N/AC catalyst A achieves the catalytic cycling without the need of adding the additional phosphine ligand, and has high activity and C₂ selectivity, achieving homogeneous of the organic phosphine ligand, thereby generating an important practical significance for reducing production costs, reducing environmental pollution, and achieving green catalysis.

### Embodiment 9

A 4-acetyloxy-2-methyl-2-butenal is prepared from catalyzing hydroformylation of a but-3-ene-1,2-diyl diacetate.

Compared with the embodiment 2, a difference is that the Rh@P—N/AC catalyst A is replaced by the Rh@P—N/AC catalyst B. And then, the reaction product is sampled to analyze, finding that a conversion rate of the raw materials reaches 98.0% and C₂ selectivity reaches 79.3%.

### Embodiment 10

A 4-acetyloxy-2-methyl-2-butenal is prepared from catalyzing hydroformylation of a but-3-ene-1,2-diyl diacetate.

Compared with the embodiment 2, a difference is that the Rh@P—N/AC catalyst A is replaced by the Rh@P—N/AC catalyst C. And then, the reaction product is sampled to analyze, finding that a conversion rate of the raw materials reaches 97.9% and C₂ selectivity reaches 77.9%.

### Embodiment 11

A 4-acetyloxy-2-methyl-2-butenal is prepared from catalyzing hydroformylation of a but-3-ene-1,2-diyl diacetate.

Compared with the embodiment 2, a difference is that the Rh@P—N/AC catalyst A is replaced by the Rh@P—N/AC catalyst D. And then, the reaction product is sampled to analyze, finding that a conversion rate of the raw materials reaches 98.4% and C₂ selectivity reaches 78.1%.

### Embodiment 12

A 4-acetyloxy-2-methyl-2-butenal is prepared from catalyzing hydroformylation of a but-3-ene-1,2-diyl diacetate.

Compared with the embodiment 2, a difference is that the Rh@P—N/AC catalyst A is replaced by the Rh@P—N/AC catalyst E. And then, the reaction product is sampled to analyze, finding that a conversion rate of the raw materials reaches 98.7% and C₂ selectivity reaches 76.7%.

### Embodiment 13

A 4-acetyloxy-2-methyl-2-butenal is prepared from catalyzing hydroformylation of a but-3-ene-1,2-diyl diacetate.

Compared with the embodiment 2, a difference is that the Rh@P—N/AC catalyst A is replaced by the Rh@P—N/AC catalyst F. And then, the reaction product is sampled to analyze, finding that a conversion rate of the raw materials reaches 92.3% and C₂ selectivity reaches 78.4%.

### Embodiment 14

A 4-acetyloxy-2-methyl-2-butenal is prepared from catalyzing hydroformylation of a but-3-ene-1,2-diyl diacetate.

Compared with the embodiment 2, a difference is that the Rh@P—N/AC catalyst A is replaced by the Rh@P—N/AC catalyst G. And then, the reaction product is sampled to analyze, finding that a conversion rate of the raw materials reaches 91.2% and C₂ selectivity reaches 76.8%.

### Test Example

A cycling performance of a catalyst is tested.

A 4-acetyloxy-2-methyl-2-butenal is prepared form catalyzing hydroformylation of a but-3-ene-1,2-diyl diacetate by using the Rh@P—N/AC catalyst A and the test includes the following steps:
using the Rh@P—N/AC catalyst A to perform a first cycle, realized by centrifuging the Rh@P—N/AC catalyst A and then washing with water (10 mL each time for 3 times) and with toluene (10 mL each time for 3 times), thereafter drying the Rh@P—N/AC catalyst A at 60° C. In the first cycle, a conversion ration of the raw materials is 99.1% and C₂ selectivity reaches 80.2%.

And then, the Rh@P—N/AC catalyst A is used to perform a second cycle. In the second cycle, a conversion ration of the raw materials is 98.6% and C₂ selectivity reaches 79.9%.

Finally, the Rh@P—N/AC catalyst A is used to perform a third cycle. In the third cycle, a conversion ration of the raw materials is 98.2% and C₂ selectivity reaches 79.8%.

The above are only the illustrated embodiments of the disclosure. It should be pointed out that for those skilled in the related art, several improvements and embellishments can be made without departing from the principles of the disclosure. These improvements and embellishments should also be considered as the scope of the protection of the disclosure.

What is claimed is:

1. A supported hydroformylation catalyst, wherein a structure expression of the supported hydroformylation catalyst is Rh@P—N/AC, Rh represents rhodium (Rh) atoms that are configured to be an active center of the supported hydroformylation catalyst, and P—N/AC represents carriers of the supported hydroformylation catalyst comprising an AC carrier and a P—N carrier; and
wherein the AC carrier in the P—N/AC is an activated carbon; and the P—N carrier in the P—N/AC is a phosphine-nitrogen (P—N) organic ligand with one structural formula selected from the group consisting of the following structural formulas I-IV:

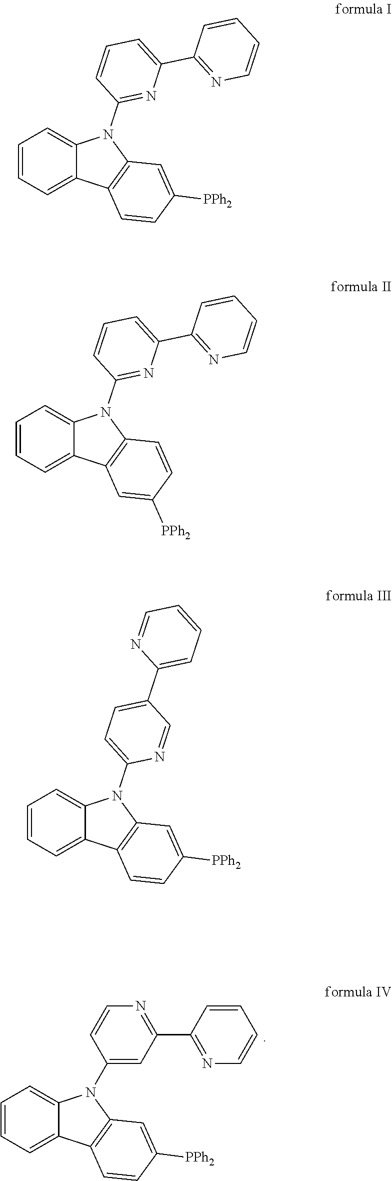

2. The supported hydroformylation catalyst as claimed in claim 1, wherein a particle size of each of the Rh atoms is in a range of 1-3 nanometers (nm); a supported capacity of the Rh atoms is in a range of 2% to 5%; and the activated carbon is powdered with a specific surface in a range of 800 square meters per gram (m²/g) to 1,000 m²/g.

3. The supported hydroformylation catalyst as claimed in claim 1, wherein a preparation method of the P—N organic ligand comprises the following steps:
performing a coupled reaction on a 2-bromocarbazole and a bipyridine bromide to obtain an N-bipyridine carbazole intermediate, and then performing a couple reaction on the N-bipyridine carbazole intermediate and a chlorodiphenyl phosphine to obtain the P—N organic ligand.

4. A preparation method of the supported hydroformylation catalyst as claimed in claim 1, comprising the following steps:
step 1, performing pre-coordination on the P—N organic ligand and a Rh precursor to obtain a mixed solution;
step 2, adding the activated carbon to the mixed solution obtained in the step 1, performing ultra-sonication on the mixed solution added with the activated carbon to disperse the carriers and obtaining a solid catalyst sample by impregnation, and rotary evaporating and drying the solid catalyst sample to obtain a solid sample; and
step 3, performing hydrogenation reduction and roasting carbonization on the solid sample obtained in the step 2 at a temperature of 500 degrees Celsius (° C.) to 700° C. with a heating rate of 5-10° C. per minute (° C./min), thereby obtaining the supported hydroformylation catalyst.

5. The preparation method as claimed in claim 4, wherein in the step 1, a molar ratio of Rh element in the Rh precursor to the P—N organic ligand is (0.5-10): 1.

6. The preparation method as claimed in claim 4, wherein the Rh precursor is a rhodium chloride trihydrate or a dicarbonylacetylacetonato rhodium.

7. The preparation method as claimed in claim 5, wherein reaction conditions of the pre-coordination in the step 1 comprise: a room temperature, an atmospheric pressure, and a reaction time in a range of 2-5 hours (h).

8. The preparation method as claimed in claim 5, wherein a time for the roasting carbonization is 2 h.

9. A method for synthesizing a 4-acetyloxy-2-methyl-2-butenal, comprising:
performing hydroformylation of a but-3-ene-1,2-diyl diacetate by using the Rh@P—N/AC supported hydroformylation catalyst as claimed in claim 1 to obtain the 4-acetyloxy-2-methyl-2-butenal.
